(19) United States
(12) Patent Application Publication
(10) Pub. No.: US 2026/0263223 A1
(43) Pub. Date: Sep. 10, 2026

(54) JOINT SPACERS

(71) Applicant: DSM IP ASSETS B.V., Maastricht (NL)

(72) Inventors: Lawrence P. LAVELLE, Columbia, MD (US); Jacob RIFFEY, Exton, PA (US); Albert LLENAS, Columbia, MD (US)

(21) Appl. No.: 18/868,068

(22) PCT Filed: May 24, 2023
(86) PCT No.: PCT/US2023/023332

§ 371 Date: Nov. 21, 2024

Related U.S. Application Data
(60) Provisional application No. 63/345,245, filed on May 24, 2022.

(51) Int. Cl. A61F 2/30; A61F 2/38

(52) U.S. Cl. CPC A61F 2/30965; A61F 2/30721; A61F 2/3872; A61F 2002/30011; A61F 2002/30014; A61F 2002/30112; A61F 2002/30324; A61F 2002/30448; A61F 2002/30754; A61F 2002/30971

(57) ABSTRACT

Joint spacers are disclosed. In an embodiment, a joint spacer comprises a foam (1), a first articular surface (3) and a second articular surface (4). The foam has a first porosity at a first location and a second porosity, less than the first porosity, at a second location. In an embodiment, the first porosity is more proximate a first external surface (5) of the joint spacer and the second location is more proximate a second external surface (6) of the joint spacer.

### FIELD

The disclosed inventions relate to joint spacers comprising foams, methods of forming joint spacers comprising foams, and methods of treating patients with such joint spacers.

### BACKGROUND

Joint spacers are medical devices that are common in the orthopedic field. A joint spacer separates and supports the bones of a joint to provide relief from pain or promote healing in a desired configuration. Joint spacers may also act as soft tissue replacement for damaged or removed tissues.

Joint spacers may be used to treat rotator cuff tears, such as massive, irreparable rotator cuff tears. The goal of such joint spacers is to restore the biomechanics of the glenohumeral joint through the depression of the humeral head and reduction of subacromial friction during shoulder abduction. Additionally, the joint spacer can be delivered arthroscopically to the subacromial space via a cannula, where it unfurls or is inflated. It therefore offers a relatively quick and potentially lower risk procedure to relieve pain.

One example of a joint spacer used in the subacromial space is the InSpace biodegradable subacromial spacer from Stryker. The device is a biodegradable and designed to restore the subacromial space without requiring sutures or fixation devices.

Further applications of joint spacers are in the knee or hip, such as an artificial meniscus in the knee. The geometry of a joint spacer may vary based on the specific geometry. For example, a joint spacer for use as an artificial meniscus may take the general shape of the biological meniscus.

### SUMMARY

There are several potential disadvantages with current joint spacers. First, difficulties may be experienced in securing the joint spacer in place. Second, biodegradable spacers may be less preferred than biostable materials due to the biological effects of the degradation products within the body and the short-term effect of the intervention. Third, discomfort or inflammation may be experienced due to the friction experienced between the head of the joint and the joint spacer.

In order to permanently fix a joint spacer in the body, a suture or other method of securing the joint spacer may be required. The alternative is to use a balloon or other expandable material, which may still move out of place after implantation. Attaching a joint spacer with sutures has its own disadvantages in that rubbing of sutures against the joint spacer may develop tears in the joint spacer over time. Accordingly, a joint spacer that is able to be fixed in place for long-term implantation without tearing may be desired.

In accordance with an embodiment of the invention, the joint spacer comprises a biostable scaffold that allows for tissue ingrowth. The biostable scaffold is preferably of sufficient strength at certain locations to allow for long term fixation by suture without tearing. In embodiments, this is achieved by a compression and thermal setting technique that results in a variation of porosity of the foam that correlates with its thickness. Porosity is defined herein as the percentage of void space. In an embodiment, the thickness of the foam decreases in thickness from a first location to a second location the joint spacer. Thereby, the porosity of the foam is greater at the first location than the second location. In embodiment, the porosity of the foam is less at the second location than at the first location due to increased compression of the foam at the second location. The increased compression, and thereby decreased porosity, at the second location increases the tear strength of the foam and better allows for the securing of the joint spacer via suture or other fixation method.

In accordance with an embodiment of the invention, a joint spacer comprises a first, joint-facing side and a second, bone-facing side opposite the first side. The first side may be configured for tissue ingrowth via the presence of a foam. The second side may be configured to avoid irritation or wear on a surface, such as by comprising a different material than the first surface. In an embodiment, the first side and/or the second side are planar surfaces.

In an embodiment, the first side is a first articular surface and the second side is a second articular surface. In the context of the joint spacer of the invention, an articular surface is a surface of the joint spacer that faces an articular surface of a joint. For example, in the context of a meniscus implant, the first articular surface may be configured to face the articular surface of the tibial plateau and the second articular surface may be configured to face the articular surface of the femoral condyle. In an embodiment the first side of the joint spacer is opposite the second side of the joint spacer.

In accordance with a further embodiment of the invention, a joint spacer comprises a foam. The foam is porous and decreases in porosity from a first location and a second location. In an embodiment, the first location is a first outward facing surface and one end of the periphery of the joint spacer and the second location is at a second outward facing surface at the opposite end of the periphery of the joint spacer. In an embodiment, the first outward facing surface of the foam is not an articular surface. In an embodiment, the second outward facing surface of the foam is not an articular surface.

The first articular surface and or the second articular surface may be made of a different material than the foam or comprise a surface coating contained within the pores of a foam. In an embodiment, an articular surface of the joint spacer comprises a polyurethane sheet. In an embodiment, the polyurethane sheet is secured to the foam by physical entanglement with the foam. In an embodiment, a thin layer of collagen is coated on the surface of the polyurethane sheet or the foam to create a boundary layer on the surface.

In further embodiments, the joint spacer can be fabricated with reinforcing members to assist fixation and/or increase mechanical strength. The reinforcing members may be made of a different material than the foam. The reinforcing members may be comprised of sutures, tapes, ribbons, textiles, or solid configurations such as bone screws or molded anchors. In an embodiment, the reinforcing members are made from an UHMWPE fiber tape commonly used in orthopedic repairs. In an embodiment, the reinforcing member is positioned such that it enables attachment of the joint spacer to the bone surface, such as the humeral head in the shoulder, to fix the spacer at a specific position within the interior of the joint.

The joint spacers disclosed herein may exhibit improvements in tissue ingrowth or repair, a reduction in inflammation, increased duration of treatment, improved dimensional stability, ease in administration, more economical fabrication or administration, a reduction in complications, improved placement stability, improved suture retention, a reduction in tearing, or improved patient comfort.

### BRIEF DESCRIPTION OF FIGURES

FIG. 1 is a side cross-sectional view of a joint spacer according to an embodiment of the invention.

FIG. 2 is a photograph of a mold capable of forming a meniscus replacement device.

FIG. 3 is side cross-sectional view of a molding process just prior to compression of a foam.

FIG. 4 is a side cross-sectional view of a molding process after compression of a foam.

FIG. 5 is a side cross-sectional view of a joint spacer comprising two reinforcing elements in the form of sutures.

FIG. 6 is an overhead of the embodiment of FIG. 5.

FIG. 7 is a side cross-sectional view of a joint spacer comprising two reinforcing elements in the form of sutures.

FIG. 8 is a side cross-sectional view of a joint spacer comprising a textile embedded in a foam.

FIG. 9A is a side cross-sectional view of a joint spacer according to an embodiment of the invention.

FIG. 9B is a side cross-sectional view of a joint spacer according to an embodiment of the invention.

FIG. 10 is an overhead view of a first, joint-facing side of a joint spacer according to an embodiment of the invention.

FIG. 11 is side cross-sectional view of a molding process just prior to compression of a foam.

FIG. 12 is a side cross-sectional view of a molding process after compression of a foam.

### DETAILED DESCRIPTION

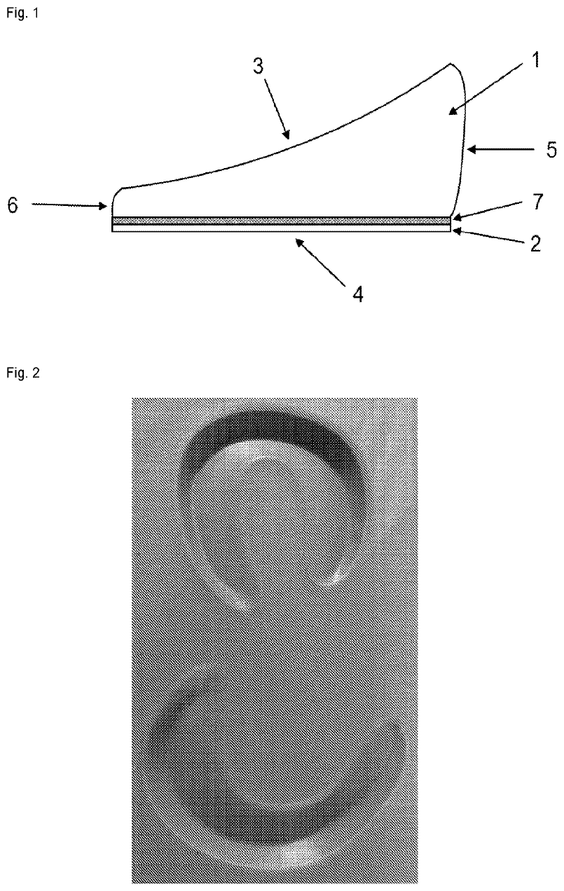

A side cross-sectional view of a joint spacer according to an embodiment of the invention is depicted in FIG. 1. In an embodiment, and as depicted in FIG. 1, the joint spacer comprises a foam 1 and a sheet 2 adhered to a surface of the foam. The depicted joint spacer comprises a first articular surface 3 and a second articular surface 4. The first articular surface 3 is configured for the articular surface of the femoral condyle and the second articular surface 4 is configured for the articular surface of the tibial plateau. As depicted, the second articular surface 4 is planar and is formed from an exterior surface of sheet 2.

The joint spacer comprises a first outward facing surface 5 and a second outward facing surface 6. The first location is present proximate first outward facing surface 5 and the second location is proximate second outward facing surface 6. The porosity of foam 1 is greatest proximate the first location, at the greatest thickness of the foam, and least at the second location, at the least thickness of the foam. In an embodiment, the variation in porosity is achieved via compression of a stock foam shape as hereinafter described.

A side cross-sectional view of a joint spacer according to an embodiment of the invention is depicted in FIG. 1. In an embodiment, and as depicted in FIG. 1, the joint spacer comprises a foam 1 and a sheet 2 adhered to a surface of the foam. The joint spacer comprises a joint-facing side 3 and a bone-facing side 4.

The joint-facing side comprises a convex protrusion 5 formed from the foam. The joint-facing side also comprises a peripheral portion 6A, 6B formed from the foam. The depicted foam has a porosity that decreases radially from the thickest part of the convex protrusion and remains constant through the peripheral portion. As depicted, the peripheral portion is planar. In an embodiment, the variation in porosity is achieved via compression of a stock foam shape as hereinafter described.

The joint spacer comprises a foam. In an embodiment, the foam is an open-cell foam. The foam is biocompatible. In an embodiment, the foam is biostable. In an embodiment, the foam is biodegradable. In an embodiment, the foam is elastomeric. In an embodiment, the foam comprises a polyurethane. In an embodiment, the foam comprises a thermoset polyurethane.

In an embodiment, the foam has a continuous and interconnected void phase. A continuous and interconnected void phase is a continuous network of structure defining a void space therein, wherein said void space comprises a plurality of interconnected pores forming a continuous network of intercommunicating passageways. In an embodiment, the continuous and interconnected void phase extends from a surface of the foam. In an embodiment, the continuous and interconnected void phase extends through the foam such that the foam has fluid permeability from one surface to another. In an embodiment the foam comprises an internal continuous and interconnected void phase and an external skin layer. In an embodiment, the continuous and interconnected void phase is formed by reticulation as hereinafter described.

In an embodiment, the foam is coated with a material to encourage cellular ingrowth or proliferation. In an embodiment, such coating comprises collagen, fibronectin, elastin, hyaluronic acid, or a mixture thereof. In an embodiment, a thin layer of collagen is coated on a surface of the joint spacer to create a boundary layer on the surface. In an embodiment, a surface of the joint spacer is coated with a bioactive agent or a coating comprising a bioactive agent. In an embodiment, the foam is coated with a bioactive agent or a coating comprising a bioactive agent.

In an embodiment, the foam is as described in U.S. Pat. Nos. 7,803,395 or 9,050,176, which are each hereby incorporated by reference in their entirety.

In an embodiment, a plurality of interconnected pores have an average diameter or other largest transverse dimension of at least about 50 μm. In an embodiment, the void space comprises from about 70% to about 99% of the volume of the foam in its uncompressed state.

The foam is compressed from its neutral state in a plurality of locations. In an embodiment, the foam comprises an uncompressed location. In an embodiment, the foam is compressed in all locations. In an embodiment, the foam has a density ratio from its most compressed location to its least compressed location of from 15:1, 12:1, 10:1 or 9:1 to 2:1, 3:1, 4:1, 5:1, or 6:1. In an embodiment, the foam has a density ratio from its most compressed location to least compressed location of about 8:1.

In an embodiment, the bulk density of the foam, prior to any compression, may be from about 0.008 g/cc to about 0.96 g/cc. In another embodiment, the bulk density is from 0.25 to 0.75 g/cc. In another embodiment, the bulk density may be from about 0.016 g/cc to about 0.56 g/cc. In another embodiment, the bulk density may be from about 0.008 g/cc to about 0.15 g/cc. In another embodiment, the bulk density may be from about 0.008 g/cc to about 0.127 g/cc. In another embodiment, the bulk density may be from about 0.008 g/cc to about 0.288 g/cc. In another embodiment, the bulk density may be from about 0.016 g/cc to about 0.115 g/cc. In another embodiment, the bulk density may be from about 0.024 g/cc to about 0.104 g/cc. Bulk density is as measured pursuant to the test method described in ASTM Standard D3574.

In an embodiment, a surface of the joint spacer comprises a sheet. In an embodiment, the sheet comprises a planar surface. The sheet may cover all or part of a surface of the joint spacer. In an embodiment, the sheet is adhered to the foam by physical entanglement of polymer chains. In an embodiment, the sheet is configured to contact the articular surface of the femoral condyle.

In an embodiment, the sheet provides a smooth external surface on the joint spacer that limits friction on a tissue surface, such as bone. Accordingly, in an embodiment the sheet is present on a bone-facing side of the joint spacer. In an embodiment, the sheet has a hardness of from Shore 50 A, 60 A, 70 A or 75 A to 80 D, 75 D, 70 D, 60 D, or 50 D. The sheet may also have advantages in further improving the suture retention of the joint spacer, such as at its periphery. In an embodiment, the sheet extends across an entire surface of the foam. In an embodiment, the sheet at least partially covers a surface of the foam.

In an embodiment the sheet comprises a polyurethane. In an embodiment, the sheet comprises a thermoplastic polyurethane. In an embodiment, the sheet is formed by casting a film comprising the polyurethane dissolved in a solvent and then evaporating the solvent. In an embodiment, the sheet is formed by molding or extrusion. In an embodiment, the sheet has a thickness of from 10 microns to 1 mm. In an embodiment, the sheet has a thickness of from 50 to 500 microns.

The joint spacer may exhibit resilience, that is it is able to recoil or spring back into shape after being bent, stretched, or compressed. Such feature may allow for targeted delivery and release at a surgical site using minimally invasive means, such as by, e.g., catheter, endoscope, arthroscope, laparoscope, cystoscope or syringe. Upon deliver at the target site, the joint spacer may substantially regain its shape.

An increased density of the foam at, for example, a peripheral location of the joint spacer allows for increased suture tear resistance over the foam in its uncompressed state. Referring again to FIG. 1, the second location (proximate second outward facing surface 6) may offer increased suture tear resistance and thus an advantageous location for attaching the joint spacer to the anatomy of a patient as a result of the foam having lower porosity and thus generally higher compression at the location. In an embodiment, one or more features to secure the joint spacer, such as holes or embedded sutures are present, preferably proximate the second location.

The foam is formed from a matrix. In an embodiment, the matrix is a polymer. In an embodiment, the matrix is formed by a reaction of a mixture comprising: (i) a polyol, and (ii) an isocyanate component. In an embodiment, the mixture further comprises a chain extender. In an embodiment, the matrix is thermoset. In an embodiment, the matrix is thermoplastic. In an embodiment, elastomeric matrix is free of allophanate and biuret linkages.

An embodiment relates to a process for preparing an elastomeric matrix comprising a continuous network of intercommunicating passageways, said process comprising: (a) synthesizing a polycarbonate polyurethane foam comprising a plurality of cell walls defining a plurality of pores therein by reacting a mixture comprising: (i) a polycarbonate polyol, (ii) an isocyanate component, and (iii) a blowing agent for forming said plurality of pores; and (b) igniting a combustible gas to remove at least about 40% of said plurality of cell walls to form said continuous network of intercommunicating passageways.

In an embodiment, the foam comprises a polyurethane. In an embodiment, the sheet comprises a polyurethane. The polyurethane comprises the reaction product of an isocyanate and a polyol. In an embodiment, the polyurethane comprises the reaction product of a diisocyanate, a polymeric aliphatic diol, and optionally a chain extender. In an embodiment, the polyurethane consists of the reaction product of a diisocyanate, a polymeric aliphatic diol, and a chain extender diol. In an embodiment, the polyurethane is linear.

By a reaction product it is meant that the isocyanate and polyol are engaged in a simultaneous or sequential chemical reaction. For example, a reaction product of a diisocyanate, a polymeric aliphatic diol, and a chain extender diol is formed i) when the diisocyanate, polymeric aliphatic diol, and chain extender diol are all reacted together in a single solution, or ii) when a pre-polymer is first formed by reacting the diisocyanate and the polymeric aliphatic diol, and then this prepolymer is subsequently reacted with a chain extender diol.

The components of an exemplary polyurethane comprising a diisocyanate, a polymeric aliphatic diol, and a chain extender will now be described. Such components could be present in the foam, the sheet, or both.

### Diisocyanate

The polyurethane comprises the residue of a diisocyanate. In an embodiment, the diisocyanate is aliphatic. In an embodiment, the diisocyanate is aromatic. In an embodiment, the diisocyanate comprises 2,4′-diphenylmethane diisocyanate, 4,4′-diphenylmethane diisocyanate (MDI), 2,4-toluene diisocyanate, 2,6-toluene diisocyanate, 1,4-phenylene diisocyanate, hexamethylene diisocyanate (HDI), tetramethylene-1,4-diisocyanate, cyclohexane-1,4-diisocyanate, dicyclohexylmethane-4,4′-diisocyanate (HMDI), isophorone diisocyanate (IPDI), or a mixture thereof. In an embodiment, the diisocyanate comprises hexamethylene diisocyanate, dicyclohexylmethane 4,4′-diisocyanate, isophorone diisocyanate, or a mixture thereof. In an embodiment, the diisocyanate consists of hexamethylene diisocyanate, dicyclohexylmethane 4,4′-diisocyanate, isophorone diisocyanate, or a mixture thereof. In an embodiment, the diisocyanate comprises 4,4′-diphenylmethane diisocyanate (MDI), 2,4-toluene diisocyanate, 2,6-toluene diisocyanate, or 1,4-phenylene diisocyanate. In an embodiment, the diisocyanate consists of 4,4′-diphenylmethane diisocyanate (MDI), 2,4-toluene diisocyanate, 2,6-toluene diisocyanate, 1,4-phenylene diisocyanate, or a mixture thereof. In an embodiment, the diisocyanate comprises 2,4′-diphenylmethane diisocyanate, 4,4′-diphenylmethane diisocyanate, or a mixture thereof.

In an embodiment, the molecular weight of the diisocyanate is from 100 to 500 g/mol. In an embodiment, the molecular weight of the diisocyanate is from 150 to 260 g/mol.

In an embodiment, the formulation from which the polyurethane is formed comprises at least 10 wt %, at least 20 wt %, at least 25 wt %, at least 30 wt %, at least 35 wt %, or at least 40 wt % of a diisocyanate, based on the total weight of the formulation. In an embodiment, the formulation from which the polyurethane is formed comprises at most 50 wt %, at most 40 wt %, at most 35 wt %, at most 30 wt %, at most 25 wt %, or at most 20 wt % of a diisocyanate, based on the total weight of the formulation. In an embodiment, the polyurethane comprises at least 10 wt %, at least 20 wt %, at least 25 wt %, at least 30 wt %, at least 35 wt %, or at least 40 wt % of the residue of a diisocyanate, based on the polyurethane. In an embodiment, the polyurethane comprises at most 50 wt %, at most 40 wt %, at most 35 wt %, at most 30 wt %, at most 25 wt %, or at most 20 wt % of the residue of a diisocyanate, based on the total weight of the polyurethane.

### Polymeric Polyol

The polyurethane comprises the residue of a polymeric polyol. In an embodiment, the polyurethane comprises the residue of a polymeric diol. A polymeric polyol comprises at least two OH groups and a backbone. The OH groups may be directly attached to the backbone or may be separated by a linker. For example, a hydroxyalkyl terminated polydimethylsiloxane (carbinol terminated) is a polymeric diol.

In an embodiment, the polymeric polyol comprises an aliphatic polymeric polyol. In an embodiment, the polymeric polyol comprises an aromatic polymeric polyol.

In an embodiment, the polymeric polyol comprises a poly(alkylene oxide), a polycarbonate, a polysiloxane, a random or block copolymer thereof, or a mixture thereof. In an embodiment, the polymeric polyol comprises a poly(alkylene oxide), a polycarbonate, a random or block copolymer thereof, or a mixture thereof. In an embodiment, the polymeric polyol comprises C₂-C₁₆ fluoroalkyl or C₂-C₁₆ fluoroalkyl ether.

In an embodiment, a difunctional polymeric polyol cannot, on its own, induce sufficient crosslinking for the polyurethane foam. Therefore, a higher functionality polyol, such as a triol or tetraol is used.

In an embodiment, the polymeric polyol comprises a poly(ethylene oxide) diol, a poly(propylene oxide) diol, a poly(tetramethylene oxide) diol, a poly(isobutylene) diol, a polyester diol, for example a polyester diol formed from adipic acid or isophtalic acid and a monomeric diol, an alkane diol, such as a hydrogenated polybutadiene diol or a polyethylene diol, a poly(hexamethylene carbonate) diol, a poly(polytetrahydrofuran carbonate) diol, a polysiloxane diol, a random or block copolymer diol of poly(ethylene oxide) and poly(propylene oxide), a random or block copolymer diol of poly(ethylene oxide) and poly(tetramethylene oxide), a random or block copolymer diol of poly(ethylene oxide) and a polysiloxane, or a mixture thereof.

In an embodiment, the polymeric polyol comprises a polycarbonate diol. In an embodiment, the polymeric aliphatic diol comprises a polycarbonate diol that comprises a poly(hexamethylene carbonate) diol or a poly(polytetrahydrofuran carbonate) diol. In an embodiment, the polymeric diol comprises a polycarbonate diol having a Mn of at least 500 g/mol, at least 750 g/mol, at least 1000 g/mol, or at least 1500 g/mol. In an embodiment, the polymeric aliphatic diol comprises a polycarbonate diol having a Mn of at most 10,000 g/mol, at most 7500 g/mol, at most 5000 g/mol, at most 4000 g/mol, at most 3000 g/mol, or at most 2500 g/mol.

In an embodiment, the polymeric polyol comprises a polysiloxane diol, a polycarbonate diol, or a poly(tetramethylene oxide) diol. In an embodiment, the polymeric aliphatic diol consists of a polysiloxane diol, a polycarbonate diol, a poly(tetramethylene oxide) diol, or a mixture thereof. In an embodiment, the polymeric diol comprises a mixture of two or more of a polysiloxane diol, a polycarbonate diol, or a poly(tetramethylene oxide) diol. In an embodiment, the polymeric diol consists of a mixture of two or more of a polysiloxane diol, a polycarbonate diol, or a poly(tetramethylene oxide) diol.

In an embodiment, the polymeric polyol has a Mn of at least 200 g/mol, at least 250 g/mol, at least 300 g/mol, at least 400 g/mol, or at least 500 g/mol, at least 600 g/mol, at least 700 g/mol, at least 800 g/mol, at least 900 g/mol, or at least 1000 g/mol. In an embodiment, the polymeric aliphatic diol has a Mn of at most 10,000 g/mol, at most 8500 g/mol, at most 6000 g/mol, at most 5000 g/mol, at most 4000 g/mol, at most 3000 g/mol, at most 2000 g/mol, or at most 1500 g/mol.

In an embodiment, the polyurethane is formed from a formulation that comprises at least 20 wt %, at least 30 wt %, at least 40 wt %, at least 50 wt %, or at least 60 wt % of a polymeric aliphatic polyol, based on the total weight of the formulation. In an embodiment, the polyurethane is formed from a formulation that comprises at most 80 wt %, at most 70 wt %, at most 60 wt %, or at most 50 wt % of a polymeric polyol, based on the total weight of the formulation. In an embodiment, the polyurethane comprises at least 20 wt %, at least 30 wt %, at least 40 wt %, at least 50 wt %, or at least 60 wt % of the residue of a polymeric aliphatic polyol, based on the total weight of the polyurethane. In an embodiment, the polyurethane comprises at most 80 wt %, at most 70 wt %, at most 60 wt %, or at most 50 wt % of the residue of a polymeric polyol, based on the total weight of the polyurethane.

### Chain Extender

In an embodiment, the polyurethane comprises the residue of a chain extender. The chain extender is a low molecular weight polyol, typically a diol. A triol or higher functional chain extender may be used if cross-linking is desired. In an embodiment, the chain extender is a diol and the polyurethane is a thermoplastic. In an embodiment, the chain extender is a diol and the polyurethane is a thermoset. In an embodiment, the chain extender is a diol and the polyurethane is a thermoplastic.

In an embodiment, the polyurethane comprises the residue of a chain extender diol. A chain extender diol is a non-polymeric diol having a molecular weight of 500 g/mol or less. In an embodiment, the chain extender diol is an alkane diol having from 2 to 20 carbon atoms, wherein one or more carbon atoms may be substituted with oxygen, silicon, phosphorous, or sulfur. In an embodiment, the chain extender diol is an alkane diol having from 2 to 20 carbon atoms, wherein one or more carbon atoms may be substituted with oxygen or silicon. In an embodiment, the chain extender diol is an alkane diol having from 2 to 20 carbon atoms, wherein one or more carbon atoms may be substituted with oxygen. In an embodiment, the chain extender diol is an unsubstituted alkane diol having from 2 to 20 carbon atoms.

An unsubstituted alkane diol is a diol consisting of single-bonded carbon and hydrogen atoms and two OH groups. A substituted alkane diol would be an alkane diol but for the substitution of one or more carbon atoms with another atom, such as oxygen or silicon, while still retaining at least two carbon atoms. Examples of unsubstituted alkane diols are ethylene glycol, propanediol, butanediol, pentanediol, 1,4-cyclohexanedimethanol, and the like. Examples of substituted alkane diols are diethylene glycol, dipropylene glycol, 1,3-bis(4-hydroxybutyl)tetramethyldisiloxane (BHTD), 1,3-bis(hydroxypropyl)tetramethyldisiloxane, 1,3-bis(3-hydroxyisobutyl)tetramethyldisiloxane, 3-ethoxy-1,2-propanediol, or 2,2′-Thiodiethanol.

In an embodiment, the chain extender diol comprises ethylene glycol, diethylene glycol, propylene glycol, dipropylene glycol, 1,3-propanediol, 1,2-butanediol, 1,3-butanediol, 1,4-butanediol, 2,3-butanediol, 1,2-pentanediol, 1,3-pentanediol, 1,4-pentanediol, 1,5-pentanediol, 1,3-hexanediol, 1,4-hexanediol, 1,5-hexanediol, 1,6-hexanediol, 2,4-hexanediol, 2,5-hexanediol, 1,2-octanediol, 1,3-octanediol, 1,4-octanediol, 1,5-octanediol, 1,6-octanediol, 1,7-octanediol, 1,8-octanediol, 2,7-octanediol, neopentyl glycol, 1,2-cyclohexanediol, 1,3-cyclohexanediol, 1,4-cyclohexanediol, 1,2-cyclohexanedimethanol, 1,3-cyclohexanedimethanol, 1,4-cyclohexanedimethanol, or 1,1-cyclohexanedimethanol. In an embodiment, the chain extender diol comprises ethylene glycol, propylene glycol, 1,3-propanediol, 1,2-butanediol, 1,3-butanediol, 1,4-butanediol, 2,3-butanediol, 1,2-pentanediol, 1,3-pentanediol, 1,4-pentanediol, 1,5-pentanediol, 1,3-hexanediol, 1,4-hexanediol, 1,5-hexanediol, 1,6-hexanediol, 2,4-hexanediol, 2,5-hexanediol, 1,2-octanediol, 1,3-octanediol, 1,4-octanediol, 1,5-octanediol, 1,6-octanediol, 1,7-octanediol, 1,8-octanediol, or 2,7-octanediol.

In an embodiment, the chain extender has a molecular weight of at least 60 g/mol, at least 70 g/mol, at least 80 g/mol, at least 90 g/mol, or at least 100 g/mol. In an embodiment, the chain extender has a molecular weight of at most 500 g/mol, at most from 400 g/mol, at most 300 g/mol, at most 200 g/mol, or at most 150 g/mol.

In an embodiment, the chain extender comprises a polyol having a functionality of at least 3. In embodiment, the chain extender comprises a monomeric triol or tetraol, or a propoxylate thereof. In an embodiment, the chain extender comprises glycerol, glycerol propoxylate, glycerol ethoxylate, 1,2,4-benzenetriol, 3-methyl-1,3,5-pentanetriol, pentaerythritol, pentaerythritol propoxylate, or pentaerythritol ethoxylate. In an embodiment, the chain extender has a molecular weight of from 90 to 500 g/mol. In an embodiment, the chain extender has a molecular weight of from 90 to 280 g/mol.

In an embodiment, the polyurethane is formed from a formulation that comprises at least 1 wt %, at least 2 wt %, at least 5 wt %, at least 8 wt %, or at least 10 wt % of a chain extender diol, based on the total weight of the formulation. In an embodiment, the polyurethane is formed from a formulation that comprises at most 20 wt %, at most 15 wt %, at most 12 wt %, at most 10 wt %, at most 8 wt %, or at most 5 wt %, of a chain extender diol, based on the total weight of the formulation. In an embodiment, the polyurethane comprises at least 1 wt %, at least 2 wt %, at least 5 wt %, at least 8 wt %, or at least 10 wt % of the residue of a chain extender diol, based on the total weight of the polyurethane. In an embodiment, the polyurethane comprises at most 20 wt %, at most 15 wt %, at most 12 wt %, at most 10 wt %, at most 8 wt %, or at most 5 wt %, of the residue of a chain extender diol, based on the total weight of the polyurethane.

### Endgroups

In an embodiment, the polyurethane comprises one or more endgroups. An endgroup is a moiety present at a terminal end of a molecule. In an embodiment, the polyurethane is linear and comprises an endgroup at each terminus of the backbone. In an embodiment, the endgroup is linear. In an embodiment, the endgroup is branched. In an embodiment, the polyurethane comprises an average of at least 0.1 endgroups, at least 0.25 endgroups, at least 0.5 endgroups, at least 1 endgroup, at least 1.5 endgroups, at least 1.8 endgroups, about 2 endgroups, or at least 2 endgroups. In an embodiment, the polyurethane comprises an average of at most 4 endgroups an average of at most 2 endgroups, or an average of at most 2 endgroups.

An endgroup may be formed by reacting a terminal isocyanate group present after forming the polymer backbone with a coreactive group on a monofunctional moiety. For instance, a terminal isocyanate group may be reacted with 1-octanol or octylamine to form a C₈ alkyl endgroup. Endgroups may also result from the inclusion of chain stoppers, such as monofunctional alcohols, in a formulation used in the formation of a polyurethane. For instance, a formulation for forming a polyurethane may comprise a diisocyanate, a polymeric aliphatic diol, a chain extender, and a monofunctional alcohol.

In an embodiment, the endgroup comprises a hydrophobic poly(alkylene oxide), a hydrophilic poly(alkylene oxide), a copolymer comprising a hydrophilic poly(alkylene oxide) and a hydrophobic poly(alkylene oxide), a polysiloxane, C₂-C₂₀ alkyl, C₂-C₁₆ fluoroalkyl, C₂-C₁₆ fluoroalkyl ether, or copolymers thereof. In an embodiment, the polysiloxane is a poly(dimethylsiloxane). In an embodiment, the hydrophilic poly(alkylene oxide) is poly(ethylene oxide). In an embodiment, the hydrophobic poly(alkylene oxide) is poly(propylene oxide) or poly(tetramethylene oxide). In an embodiment, the endgroup comprises a hydrophobic poly(alkylene oxide), a hydrophilic poly(alkylene oxide), a copolymer comprising a hydrophilic poly(alkylene oxide) and a hydrophobic poly(alkylene oxide), C₂-C₂₀ alkyl, C₂-C₁₆ fluoroalkyl, C₂-C₁₆ fluoroalkyl ether, or copolymers thereof. Such endgroups may be formed with monofunctional alcohols, including carbinols, or amines of the foregoing.

In an embodiment, the endgroup comprises C₂-C₁₆ fluoroalkyl or C₂-C₁₆ fluoroalkyl ether. Such endgroups may be formed with monofunctional alcohols or amines comprising C₂-C₁₆ fluoroalkyl or C₂-C₁₆ fluoroalkyl ether.

In an embodiment, the endgroup is formed from a monofunctional alcohol or amine comprising C₂-C₁₆ fluoroalkyl or C₂-C₁₆ fluoroalkyl ether. In an embodiment, the endgroup is formed from 1H,1H-Perfluoro-3,6-dioxaheptan-1-ol, 1H,1H-Nonafluoro-1-pentanol, 1H,1H-Perfluoro-1-hexyl alcohol, 1H,1H-Perfluoro-3,6,9-trioxadecan-1-ol, 1H,1H-Perfluoro-1-heptyl alcohol, 1H,1H-Perfluoro-3,6-dioxadecan-1-ol, 1H,1H-Perfluoro-1-octyl alcohol, 1H,1H-Perfluoro-1-nonyl alcohol, 1H,1H-Perfluoro-3,6,9-trioxatridecan-1-ol, 1H,1H-Perfluoro-1-decyl alcohol, 1H,1H-Perfluoro-1-undecyl alcohol, 1H,1H-Perfluoro-1-lauryl alcohol, 1H,1H-Perfluoro-1-myristyl alcohol, or 1H,1H-Perfluoro-1-palmityl alcohol.

In an embodiment, the endgroup is monomeric and has a molecular weight of 200 g/mol or more, 300 g/mol or more, or 500 g/mol or more. In an embodiment, the endgroup is monomeric and has a molecular weight of 1,000 g/mol or less or 800 g/mol or less. In an embodiment, the endgroup is polymeric and has a Mn of 10,000 g/mol or less, 8,000 g/mol or less, 6,000 g/mol or less, or 4,000 g/mol or less. In an embodiment, the endgroup is polymeric and has a Mn of 500 g/mol or more, 1,000 g/mol or more, or 2,000 g/mol or more.

In an embodiment, the endgroup is present in an amount of at least 0.1 wt %, at least 0.2 wt %, at least 0.3 wt %, or at least 0.5 wt %, based on the total weight of the formulation from which the polyurethane is formed. In an embodiment, the endgroup is present in an amount of at most 3 wt %, at most 2 wt % or at most 1 wt %, based on the total weight of the formulation from which the polyurethane is formed. In an embodiment, the endgroup is present in an amount of at least 0.1 wt %, at least 0.2 wt %, at least 0.3 wt %, or at least 0.5 wt %, based on the total weight of the polyurethane. In an embodiment, the endgroup is present in an amount of at most 3 wt %, at most 2 wt % or at most 1 wt %, based on the total weight of the polyurethane.

### Formation of Polyurethanes

The polyurethanes may be formed as generally known in the art. A catalyst may be employed. In an embodiment, the catalyst is stannous octoate or dibutyltin dilaurate. Amine catalysts may also be used.

In an embodiment, the polyurethane is cross-linked. In an embodiment, the foam comprises a cross-linked polyurethane. Certain polyurethanes may require cross-linking to achieve a stable foam such that the foam does not collapse. In an embodiment, foam comprises a cross-linked polyurethane and the sheet comprises a polyurethane that is not cross-linked. In order to cross-link a polyurethane, a 3+ functional compound, such as a tri-isocyanate, and/or a small quantity of an optional ingredient, such as a 3+ functional hydroxyl compound or other crosslinker having a functionality greater than 2, e.g., glycerol, is present to allow crosslinking.

### Foam Formation

Foams can be made using various procedures known in the art. Exemplary procedures for forming a foam are described in the following paragraphs.

In an embodiment, a prepolymer is first prepared by a conventional method from at least one isocyanate component (e.g., MDI) and at least one multi-functional soft segment material with a functionality greater than 2 (e.g., a polyether-based soft segment with a functionality of 3). Then, the prepolymer, optionally with a catalyst and at least one difunctional chain extender (e.g., 1,4-butanediol) are admixed in a mixing vessel to cure or crosslink the mixture. In another embodiment, crosslinking and foaming, i.e., pore formation, take place together. In another embodiment, crosslinking and foaming take place together in a mold.

In an embodiment, the polyol component is admixed with the isocyanate component and cell opener to form a first liquid. Other optional additives, such as a viscosity modifier, surfactant, chain extender and cross linker are admixed to form a catalyst batch mixture. Then, the first liquid, and the catalyst batch mixture are admixed in a mixing vessel to be foamed and cross-linked. In another embodiment, foaming and cross-linking occur simultaneously. In another embodiment, this foaming mix is poured optionally through a nozzle into a mold and allowed to rise.

Alternatively, a so-called “one-shot” approach may be used. A one-shot embodiment requires no separate prepolymer-making step. In an embodiment, the materials are admixed in a mixing vessel and then foamed and crosslinked.

In another embodiment, all of the ingredients except for the isocyanate component are admixed in a mixing vessel. The isocyanate component is then added, e.g., with high-speed stirring, and crosslinking and foaming ensue. In another embodiment, this foaming mix is poured into a mold and allowed to rise.

Another embodiment involves admixing a polyol component with an isocyanate component and other optional additives, such as a viscosity modifier, surfactant and/or cell opener, to form a first liquid. Next, a second liquid is formed by admixing a blowing agent and optional additives, such as gelling catalyst and/or blowing catalyst. Then, the first liquid and the second liquid are admixed in a mixing vessel and then foamed and crosslinked. The foaming mix is poured optionally through a nozzle into a mold and allowed to rise.

In another embodiment of the one-shot approach, the isocyanate component forms a first liquid. In one embodiment, the isocyanate component is maintained between 5 psi and 30 psi above the ambient pressure and in another embodiment, the isocyanate component is optionally maintained between 20° C. to 30° C. The polyol component is admixed with other optional additives, such as a viscosity modifier, and/or cell opener, to form a second liquid. In an embodiment, the polyol component is admixed or pre-mixed with cell opener and viscosity depressant. In another the polyol component is optionally admixed or pre-mixed with cell opener and viscosity depressant. Next, a third liquid is formed by admixing a blowing agent and a cross-liner and optionally a chain extender and optional additives, such as gelling catalyst and/or blowing catalyst and surfactants. The blowing agent is preferably water and in embodiment is distilled water. The cross-linking agent is glycerol. In one embodiment the blowing agent, water, and cross-linking agent, glycerol, are always admixed before the foaming and cross-linking reactions. Then, the first liquid, the second liquid and the third liquid are admixed in a mixing vessel and then foamed and cross-linked.

Considerations should be taken to ensure that the foaming fluid or the reacting mix is laid down on to the mold bottom surface in a linear fashion or without effective retracing of the flow paths so that it does not introduce any flow disturbances or mix up of the differently aged foaming fluid or the reacting mix coming out of the mixing vessel. In one embodiment, the foaming fluid or the reacting mix is laid down on to the mold bottom surface in a linear fashion or without effective retracing of the flow paths such that the foaming fluid or the reacting mix coming out of the mixing vessel at a later time do not introduce any flow disturbances or mix with foaming fluid or the reacting mix that came out earlier.

At the end of the foam rise, the foaming and cross-linking reaction are considered to be complete or substantially complete, thereby resulting in a matrix in the form of a foamed block or shape. In one embodiment, the matrix is then optionally subjected to additional curing at an elevated temperature. The curing ensures the utilization and/or removal of any free isocyanates and amines and/or completion or substantial completion of other un-reacted ingredients that may not have reacted during foam formation. The curing temperature can range from 70° C. to 120° C. and in other embodiments can range from 75° C. to 110° C. The curing time can range from 30 minutes to 400 minutes and in other embodiment can range from 60 minutes to 300 minutes. In one embodiment, the foamed matrix is not subjected to additional curing at an elevated temperature.

In an embodiment, the continuous and interconnected void phase is formed by reticulation. Reticulation generally refers to a process for at least partially removing cell walls, not merely rupturing or tearing them by a crushing process, which crushing process may undesirably create debris that must be removed by further processing. In another embodiment, the reticulation process substantially fully removes at least a portion of the cell walls. Reticulation may be effected, for example, by at least partially dissolving away cell walls, known variously as “solvent reticulation” or “chemical reticulation”; or by at least partially melting, burning and/or exploding out cell walls, known variously as “combustion reticulation”, “thermal reticulation” or “percussive reticulation”. In an embodiment, two reticulation steps are used, such as a first combustion reticulation followed by a second combustion reticulation. In another embodiment, combustion reticulation is followed by chemical reticulation. In another embodiment, chemical reticulation is followed by combustion reticulation.

One embodiment employs chemical reticulation, where the matrix is reticulated in an acid bath comprising an inorganic acid. Another embodiment employs chemical reticulation, where the matrix is reticulated in a caustic bath comprising an inorganic base. Another embodiment employs solvent reticulation, where a volatile solvent that leaves no residue is used in the process. Another embodiment employs solvent reticulation at a temperature elevated above 25° C. In another embodiment, a matrix comprising a polycarbonate polyurethane is solvent reticulated with a solvent selected from tetrahydrofuran (“THF”), dimethyl acetamide (“DMAC”), dimethyl sulfoxide (“DMSO”), dimethylformamide (“DMF”), N-methyl-2-pyrrolidone, or a mixture thereof. The chemical reticulation may be followed by washing or drying.

In one embodiment, combustion reticulation may be employed in which a combustible atmosphere, e.g., a mixture of hydrogen and oxygen or methane and oxygen, is ignited, e.g., by a spark. In another embodiment, combustion reticulation employs a mixture of hydrogen, oxygen and/or nitrogen.

Combustion reticulation is conducted in a pressure chamber. In an exemplary procedure, the pressure in the pressure chamber is substantially reduced from atmospheric conditions, e.g., to below about 50 -150 millitorr by evacuation for at least about 2 minutes, before, e.g., hydrogen, oxygen or a mixture thereof, is introduced. In another embodiment, the pressure in the pressure chamber is substantially reduced in more than one cycle, e.g., the pressure is substantially reduced, an unreactive gas such as argon or nitrogen is introduced then the pressure is again substantially reduced, before hydrogen, oxygen or a mixture thereof is introduced. The temperature at which reticulation occurs can be influenced by, e.g., the temperature at which the chamber is maintained and/or by the hydrogen/oxygen ratio in the chamber. In another embodiment, combustion reticulation is followed by an annealing period.

### Forming Joint Spacers

In an embodiment, the joint spacer is formed from a foam stock. In an embodiment, the foam stock comprises the shape of a rectangular prism, an elliptic cylinder, or an ovoid. The foam stock may be trimmed around the edges of the mold prior to or after to the molding step. In an embodiment, the thickness of the foam stock, such as the depth of a rectangular prism or height of an elliptic cylinder, is selected such that it is equal to the total thickness of the foam in the joint spacer. In such an embodiment, the foam will be uncompressed in at least one location.

To form the joint spacer, the foam stock is compressed into the mold in the direction of the depth of the mold. A typical mold for a meniscus implant is shown in FIG. 2.

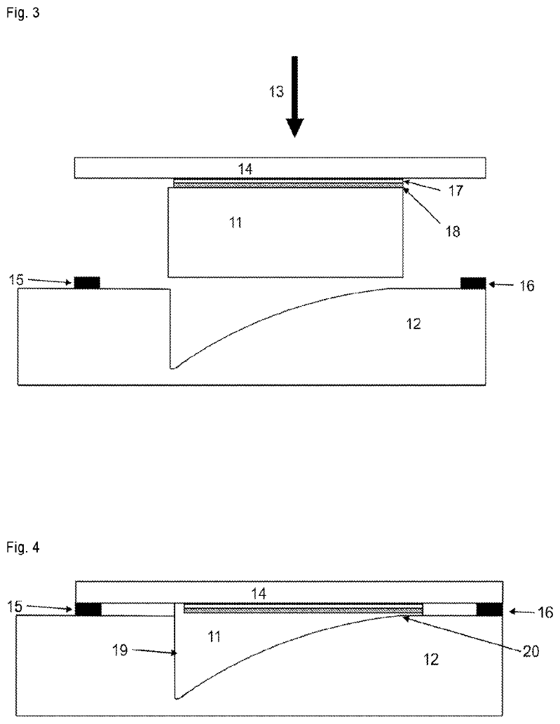

A side cross-sectional view of the molding process just prior to compression is shown in FIG. 3. Foam stock 11 has already been physically entangled to sheet 17 around textile 18 prior to compression of the foam stock 11. Sheet 17 and textile 18 do not fully cover the entire surface of foams stock 11. This may be advantageous where wear resistant properties are desired in certain areas of the joint spacer, such as in the white zone of a meniscus and tissue ingrowth facilitated by the porous foam is desired in other areas, such as the red zone of a meniscus. Foam stock 11 is then compressed into mold 12 in direction 13. Backing plate 14 may be present to facilitate a planar surface opposite the convex protrusion. Mold 12 may comprise stops 15, 16 to achieve a desired compression of the foam. The step may be performed above room temperature to facilitate softening of the foam, optimally above 120° C. to facilitate crosslinking and hydrogen bond formation within a polyurethane.

FIG. 4 depicts the process after compression of the foam. Backing plate 14 is in contact with stop 15, 16. Foam 11 has been compressed based on the depth of the mold, resulting in a variation in porosity from a first location to a second location. In an embodiment, and as depicted, the porosity of the foam decreases continually from a first location 19 to a second location 20 each proximate an opposite end of the joint spacer. In other embodiments, the foam may decrease in porosity from a first location to a second location wherein the first location and the second location are not present at opposite ends of the joint spacer. Excess foam may be trimmed after molding, such as at second location 20.

A variety of dimensions and properties can be achieved based on the manner of compressing the foam. For example, the mold surface may have variations in curvature that result in a foam having locations with varying levels of compression corresponding with the depth of the mold at such locations.

Following compression, the foam is locked in its compressed state. In the case of a thermoset foam, the temperature may be raised above 120° C. in order to lock the compression in place. The joint spacer is obtained by separating the compressed foam from the mold.

The porosity in the second location is less than the porosity at the first location. In an embodiment, the first location corresponds with the thickest portion of the joint spacer and the second location corresponds with the thinnest portion of the joint spacer. The compression in the foam, and thereby the decreased porosity, may result in increased strength at the second location. Most relevant is an increased resistance to tearing, such as by suture. This increased tear resistance allows for more permanent securement of the joint spacer by suture or other common orthopedic methods, such as anchors.

In an embodiment, and as depicted, in FIGS. 1, 3, and 4, a sheet 2, 17 is attached to the planar surface of the foam. In an embodiment, the sheet and a planar surface of the foam are treated with a solvent. Following treatment, the sheet and foam are brought into contact. This may occur prior to molding or after molding. If after molding, the bringing into contact of the foam and the sheet may occur by reinserting the foam into the mold, stacking the sheet on the foam, and setting the backing plate on top of the sheet. After evaporation of the solvent, the sheet is adhered to the planar surface of the foam. Other methods of adhering the sheet and foam are possible, such as by thermal bonding or via the use of an adhesive. In an embodiment, the joint spacer further comprises a reinforcing element. In an embodiment, the reinforcing element comprises an orthopedic suture, a textile, or an anchor. In an embodiment, the textile comprises a mesh. In an embodiment, the textile comprises a knit or woven.

The reinforcing element may be incorporated into the joint spacer in a number of ways. In an embodiment, the reinforcing element is incorporated by overmolding or bonding layers of material around the reinforcing element. In an embodiment. the position of the reinforcing element is fixed relative to the foam. In an embodiment, the reinforcing element is incorporated by passing it through the foam, such as by drilling an appropriate hole and inserting the reinforcing element. In such case, the reinforcing element is typically secured in the holes by virtue of the compression and setting of the foam. In an embodiment, the position of the reinforcing element is movable relative to the foam.

In an embodiment, a reinforcing element is secured within the joint spacer at the interface of the sheet and the foam. For example, solvent bonding may be used to secure the reinforcing element in the joint spacer by bonding together a sheet and a foam around the reinforcing element. As depicted in FIGS. 1, 3, and 4, the sheet 2, 17 and the foam 1, 11 have been solvent bonded around textile 7, 18. Similarly, the reinforcing element can be secured between multiple sheets. For example, a first reinforcing element may be secured by bonding of a first sheet to a second sheet around the first reinforcing element and a second reinforcing element may be secured by bonding of the second sheet and the foam around a second reinforcing element.

In an embodiment, the reinforcing element is inserted through the interior of the foam prior to thermal compression. For example, holes may be drilled through a foam stock and a reinforcing element inserted through the holes. Subsequently, the assembly may be compressed into a convex protrusion as previously described.

In an embodiment, the joint spacer comprises a reinforcing element secured in the convex protrusion via the bonding together of a plurality of pieces of foam. For example, a first foam stock may be bonded to a second foam stock around a reinforcing element. The assembly may then be compressed into a convex protrusion, as previously described, and optionally, later bonded to a sheet.

In an embodiment, the reinforcing element comprises a polymer cable. A polymer cable is a cable comprised of one or more polymers. In an embodiment, the polymer cable comprises high yield strength material suitable to be implanted into a human or animal body. In an embodiment, the polymer cable comprises polyethylene. In an embodiment, the polymer cable comprises a thermoplastic polyethylene. In an embodiment, the polymer cable comprises or consists of ultra-high-molecular-weight polyethylene. In an embodiment, the polymer cable comprises Dyneema Purity® Radiopaque fibers.

In an embodiment, the polymer cable comprises polymer fibers. A fiber is a long continuous filament. In an embodiment, the polymer cable consists of polymer fibers. In an embodiment, the polymer cable comprises a braid of a plurality of strands of individual polymer cables or fibers. In an embodiment, the polymer cable comprises a braid that comprises a hollow tubular braid, a solid circular braid, a spiroid braid, a flat braid, a core-sheath (sometimes called kern-mantle or core-shell) braid, or a braid-on-braid. A braid-on-braid is a core-shell construction in which a braided core is covered by another braided construction. In an embodiment, the polymer cable comprises a yarn. A yarn is a continuous strand of multiple, usually twisted, fibers. In an embodiment, the polymer cable comprises a braided, knitted, or woven cable, wherein the polymer cable comprises polymer fibers. In an embodiment, the polymer cable comprises a monofilament or a multifilament yarn. The yarn may in addition comprise other components or additives that provide some extra functional effect, such as antimicrobial or anti-inflammatory action, knotting performance, or visual contrast. In an embodiment, the polymer cable comprises a radiopaque agent. In an embodiment, the polymer cable is a suture.

In an embodiment, the polymer cable comprises a flat shape, for example, a rectangular cross-section. For example, the polymer cable may be a flat tape, wherein in a cross-sectional view, a ratio between the height and the width of the tape is less than 1, less than 0.5, less than 0.2, or less than 0.1.

In an embodiment, the reinforcing element may itself be secured to the patient's anatomy to fix the joint spacer in place in the joint. In an embodiment, the reinforcing element comprises a loop or the like that allows for another securing element, such as a separate suture, to be fixed to the reinforcing element. In an embodiment, the reinforcing member is positioned such that it enables attachment of the joint spacer to the bone surface, such as the humeral head in the shoulder, to fix the spacer at a specific position within the interior of the joint.

Various configurations of reinforcing elements may be used. In an embodiment, the joint spacer comprises two reinforcing elements that cross proximate the center of the joint spacer, each extending beyond the periphery of the joint spacer. In an embodiment, a single reinforcing element is present is present. In an embodiment, the reinforcing element in contained internally in the joint spacer.

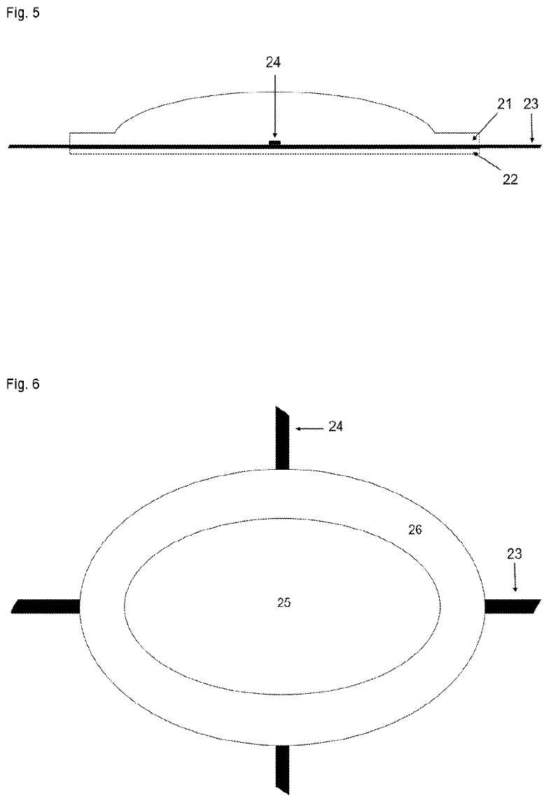

A cross-section of an embodiment of a joint spacer comprising a reinforcing element is depicted in FIG. 5. The joint spacer comprises a foam 21 forming a convex protrusion and peripheral portion. The foam is secured to a sheet 22. Sandwiched between the foam and the sheet are sutures 23 and 24, positioned perpendicularly to one another.

FIG. 6 depicts an overhead view of the same joint spacer embodiment as depicted in FIG. 5. The joint spacer comprises convex protrusion 25 and peripheral portion 26. Sutures 23 and 24 extend from the joint spacer beyond the peripheral portion 26 of the joint spacer.

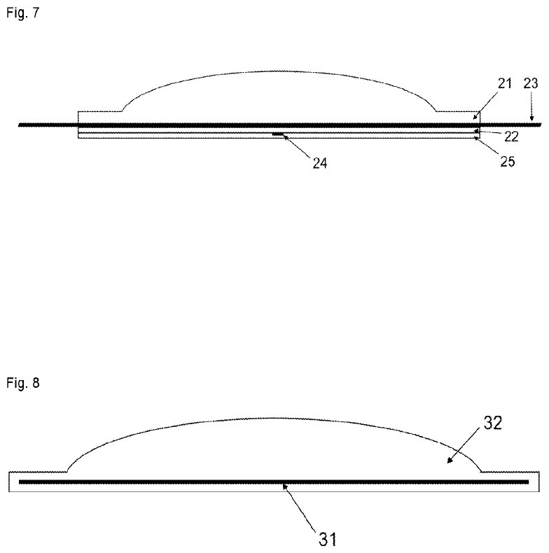

FIG. 7 depicts a cross-section of an embodiment of a joint spacer comprising two reinforcing elements. The embodiment depicted in FIG. 7 differs from the embodiment pictured in FIG. 5 in that a second sheet is present. Thus, suture 23 is sandwiched between a foam and a first sheet whereas suture 24 is sandwiched between the first sheet and a second sheet.

FIG. 8 depicts a cross-section of an embodiment of a joint spacer wherein a mesh is sandwiched between two layers of foam. The joint spacer of FIG. 8 may be assembled by providing a first foam stock and a second foam stock sandwiching a mesh. The assembly may then be compressed in a mold to create a convex protrusion and a peripheral portion, as previously described. A sheet may be subsequently attached. As depicted in FIG. 8, mesh 31 is bonded within foam 32.

In accordance with an embodiment, the foam is compressed in one or more locations, thereby decreasing its porosity and increasing its density in such locations. Such increase in density may result in a favorable increase in the suture retention strength, tensile strength, or other desirable mechanical properties in such locations, such as in the peripheral portion of the joint spacer. Depending upon the compression ratio that is used in the molding process, the porosity and resulting physical properties of the compressed sections may be designed such that the moderately compressed foam will remain open to cellular ingrowth while the more compressed foam will have a lower void fraction to achieve higher mechanical properties where desired.

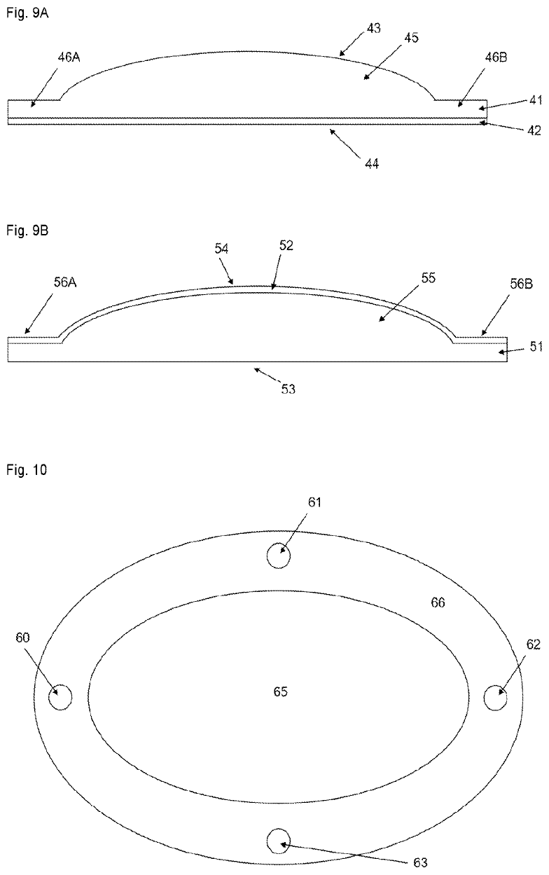

Further embodiments of joint spacers are shown in FIGS. 9A, 9B, and 10. In the embodiment depicted in FIG. 9A, the joint spacer comprises a foam 41 and a sheet 42 adhered to a surface of the foam. The joint spacer comprises a joint-facing side 43 and a bone-facing side 44.

A similar embodiment is depicted in FIG. 1B, which is a side cross-sectional view of a joint spacer according to an embodiment of the invention. As depicted in FIG. 9B, the joint spacer comprises a foam 51 and a sheet 52 adhered to a surface of the foam. The joint spacer comprises a joint-facing side 53 and a bone-facing side 54.

The joint spacers of both FIGS. 9A and 9B comprise a convex protrusion 45, 55 formed from the foam. Each also comprises peripheral portions 46A, 46B, 56A, 56B. The depicted foam has a porosity that decreases radially from the thickest part of the convex protrusion to the peripheral portion and then remains constant through the peripheral portion. As depicted in FIGS. 9A and 9B, the peripheral portion is planar.

An increased density of the foam at, for example, the peripheral portion of the joint spacer allows for increased suture tear resistance over the foam in its uncompressed state. An overhead view of the first, joint-facing side of a joint spacer according to an embodiment of the invention is shown in FIG. 10. Convex protrusion 65 comprises the shape of part of an ovoid with a peripheral portion 66 surrounding it. Peripheral portion 66 comprises a plurality of holes, 60, 61, 62, 63 which may be used to secure the joint spacer to the anatomy of a patient.

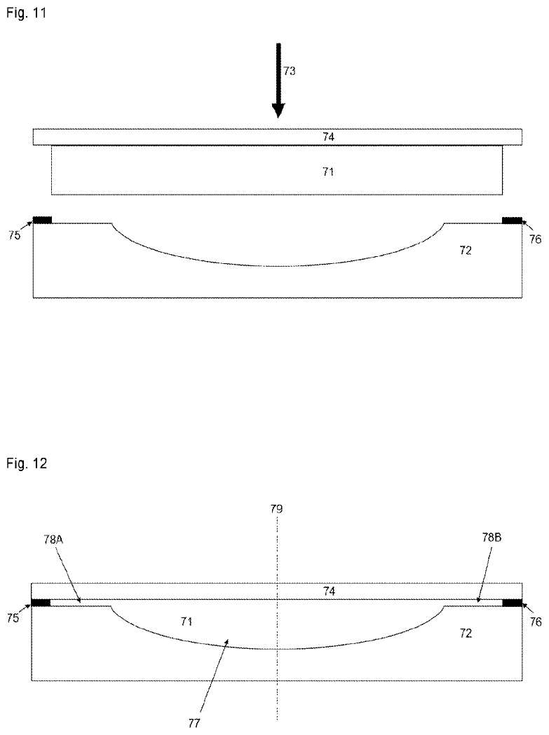

A side cross-sectional view of the molding process that may be used to form the embodiments of FIG. 9A or 9B is shown in FIG. 11. The process is depicted just prior to compression of foam stock 71. Foam stock 71 is compressed into mold 72 in direction 73. Backing plate 74 may be present to facilitate a planar surface opposite the convex protrusion. Mold 72 may comprise stops 75, 76 to achieve a desired compression of the foam in both the convex protrusion and a peripheral portion. As depicted, the peripheral portion is planar.

FIG. 12 depicts the process after compression of the foam. Backing plate 74 is in contact with stops 75, 76. Foam 71 has been compressed, thereby creating a convex protrusion 77 and peripheral portion 78A, 78B. At plane 79, the foam is uncompressed since the distance between the backing plate 74 and the deepest portion of the mold 72 is equal to the thickness of the foam stock. Peripheral portion 78A, 78B is compressed about 8 times as much as the convex protrusion at plane 79. As depicted, the thickness in the peripheral portion is ⅛^{th} of the thickness of the foam stock. Accordingly, the porosity at plane 79 is 8× the porosity and ⅛^{th} of the density of the foam in the peripheral portion.

### Applications

The disclosed joint spacers may be inserted into the joint of a patient using known means, such as arthroscopically via a cannula or alternatively via an open surgical procedure. In an arthroscopic delivery method, a joint spacer is compressed, such as by rolling it into a tube and advanced down a cannula and into the joint cavity of a patient. Upon deployment, the sides of the joint spacer are positioned appropriately and the joint spacer secured to the anatomy of the patient. This can be done by anchoring a suture positioned in the periphery of the joint spacer, such as through holes present in the periphery of the joint spacer, to a tissue of the patient. The joint spacers may be used in any suitable joint of a patient, such as the knee or shoulder. The disclosed joint spacers may have special utility in the knee joint of a patient as a meniscus repair or replacement device.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. While certain optional features are described as embodiments of the invention, the description is meant to encompass and specifically disclose all combinations of these embodiments unless specifically indicated otherwise or physically impossible.

### Description of Certain Exemplary Embodiments

The following description of exemplary embodiments of the invention is intended to aid the reader and is not intended to limit the inventions disclosed herein to only the following exemplary embodiments.

1. A joint spacer comprising a foam, the joint spacer having a first articular surface and a second articular surface, wherein the porosity of the foam varies as a function of the distance between the first articular surface and the second articular surface.

2. A joint spacer comprising a foam, the joint spacer having a first articular surface and a second articular surface, the first articular surface and the second articular defining a thickness of the joint spacer, wherein the porosity of the foam varies as a function of the thickness of the joint spacer.

3. A joint spacer comprising a foam, the joint spacer having a first articular surface and a second articular surface, the first articular surface and the second articular defining a thickness of the joint spacer, wherein a first outward facing surface is defined by the thickness of the joint spacer, the first articular surface, and the second articular surface, wherein a second outward facing surface is opposite the first outward facing surface and defined by the thickness of the joint spacer, the first articular surface, and the second articular surface, wherein the foam has a first porosity at a first location and a second porosity, less than the first porosity, at a second location, the first location being more proximate the first outward facing surface than the second outward facing surface and the second location being more proximate the second outward facing surface than the first outward facing surface.

4. The joint spacer according to the previous exemplary embodiment, wherein the first location is at the first outward facing surface and the second location is at the second outward facing surface.

5. The joint spacer according to any one of the previous exemplary embodiments, wherein the porosity of the joint spacer varies in relation to its thickness.

6. The joint spacer according to any one of the previous exemplary embodiments, wherein at a first location of the joint spacer the foam has a first porosity, at a second location of the joint spacer the foam has a second porosity, wherein the first porosity is greater than the second porosity, and wherein the thickness of the joint spacer is greater at its first porosity than at its second porosity.

7. The joint spacer according to any one of the previous exemplary embodiments, wherein at a first location of the joint spacer the foam has a first porosity, at a second location of the joint spacer the foam has a second porosity, wherein the first porosity is greater than the second porosity, and wherein the thickness of the joint spacer is greater at the first location than at the second location.

8. The joint spacer according to any one of the previous exemplary embodiments, wherein the first location is at the greatest thickness of the joint spacer and the second location is at the least thickness of the joint spacer.

9. The joint spacer according to any one of the previous exemplary embodiments, wherein the second articular surface is substantially planar.

10. The joint spacer according to any one of the previous exemplary embodiments, wherein the distance between the first articular surface and the second articular surface defines the thickness of the joint spacer, and wherein the joint spacer has uniform porosity between the first articular surface and the second articular surface at all locations of equal thickness.

11. The joint spacer according to any one of the previous exemplary embodiments, wherein the joint spacer comprises a planar portion wherein the distance between the first articular surface and the second articular surface is about constant.

12. The joint spacer according to any one of the previous exemplary embodiments, wherein the joint spacer comprises a planar portion wherein the distance between the first articular surface and the second articular surface are about constant, and wherein the porosity is substantially constant throughout the planar portion.

13. The joint spacer according to any one of the previous exemplary embodiments, wherein the foam has a porosity in the planar portion that is less than or equal to the smallest porosity in the portion of the joint spacer that is not the planar portion.

14. The joint spacer according to any one of the previous exemplary embodiments, wherein the porosity of the foam decreases radially from its center in a longitudinal direction.

15. The joint spacer according to any one of the previous exemplary embodiments, wherein the porosity of the foam decreases radially in a longitudinal direction from the thickest portion of the foam in a transverse direction.

16. The joint spacer according to any one of the previous exemplary embodiments, wherein the porosity of the foam decreases radially from its center in a longitudinal direction until the planar portion.

17. The joint spacer according to any one of the previous exemplary embodiments, wherein the porosity of the foam decreases in a longitudinal direction from the thickest portion of the foam in a transverse direction.

18. The joint spacer according to any one of the previous exemplary embodiments, wherein the foam has a porosity that is greater at its center than at its periphery.

19. The joint spacer according to any one of the previous exemplary embodiments, wherein the foam has a substantially uniform porosity in the direction of its thickness at any given radial location.

20. The joint spacer according to any one of the previous exemplary embodiments, wherein the foam is a single piece of foam.

21. The joint spacer according to any one of the previous exemplary embodiments, wherein the foam is under compression at at least one location.

22. The joint spacer according to any one of the previous exemplary embodiments, wherein the foam is under compression at all locations.

23. The joint spacer according to any one of the previous exemplary embodiments, wherein the amount of compression varies with the thickness of the joint spacer.

24. The joint spacer according to any one of the previous exemplary embodiments, wherein the foam comprises a polyurethane foam.

25. The joint spacer according to any one of the previous exemplary embodiments, wherein the polyurethane foam is an open cell polyurethane foam.

26. The joint spacer according to any one of the previous exemplary embodiments, wherein the peripheral edges of the first surface consist of the planar portion.

27. The joint spacer according to any one of the previous exemplary embodiments, wherein the planar portion comprises a plurality of holes extending from the planar portion through the joint spacer.

28. The joint spacer according to any one of the previous exemplary embodiments, wherein the planar portion comprises a sheet adhered to the foam.

29. The joint spacer according to any one of the previous exemplary embodiments, wherein the second articular surface comprises a sheet adhered to the foam.

30. The joint spacer according to any one of the previous exemplary embodiments, wherein the sheet does not comprise a foam.

31. The joint spacer according to any one of the previous exemplary embodiments, wherein the foam and the sheet are physically entangled with one another.

32. The joint spacer of any one of the previous exemplary embodiments, wherein at least 90% of the area of a surface of the sheet is entangled with the foam.

33. The joint spacer of any one of the previous exemplary embodiments, wherein the sheet is a polyurethane and the foam is a polyurethane.

34. The joint spacer of any one of the previous exemplary embodiments, wherein the foam is biostable.

35. The joint spacer of any one of the previous exemplary embodiments, wherein the foam is biodegradable.

36. The joint spacer of any one of the previous exemplary embodiments, wherein the foam comprises a thermoset polyurethane.

37. The joint spacer of any one of the previous exemplary embodiments, wherein the sheet comprises a thermoplastic polyurethane.

38. The joint spacer of any one of the previous exemplary embodiments, wherein the foam comprises a polycarbonate polyurethane.

39. The joint spacer of any one of the previous exemplary embodiments, wherein the sheet comprises a polycarbonate polyurethane.

40. The joint spacer of any one of the previous exemplary embodiments, wherein the sheet and the foam each comprise a polycarbonate polyurethane.

41. The joint spacer of any one of the previous exemplary embodiments, wherein the sheet and the foam each comprises the reaction product of an aromatic diisocyanate, a polycarbonate diol, and a chain extender.

42. The joint spacer of any one of the previous exemplary embodiments, wherein the sheet has a thickness of from 10 microns to 1000 microns.

43. The joint spacer of any one of the previous exemplary embodiments, further comprising a plurality of holes proximal the periphery of the joint spacer wherein the holes extend through the first (external) surface and the second (external) surface.

44. The joint spacer according to any one of the previous exemplary embodiments, wherein the peripheral portion comprises a plurality of holes.

45. The joint spacer according to any one of the previous exemplary embodiments, wherein the first articular surface further comprises a layer of collagen.

46. The joint spacer according to any one of the previous exemplary embodiments, wherein the second articular surface further comprises a layer of collagen.

47. The joint spacer according to any one of the previous exemplary embodiments, further comprising a reinforcing element.

48. The joint spacer according to any one of the previous exemplary embodiments, wherein the reinforcing element is embedded in the joint spacer.

49. The joint spacer according to any one of the previous exemplary embodiments, wherein the reinforcing element is fully embedded in the body of the joint spacer.

50. The joint spacer according to any one of the previous exemplary embodiments, wherein the reinforcing element passes through the body of the joint spacer and extends beyond its periphery.

51. The joint spacer according to any one of the previous exemplary embodiments, wherein the reinforcing element is secured at an interface of the foam and the sheet by a bonding of the foam and the sheet around the reinforcing element.

52. The joint spacer according to any one of the previous exemplary embodiments, wherein the reinforcing element is positioned at an interface of the foam and the sheet.

53. The joint spacer according to any one of the previous exemplary embodiments, wherein the reinforcing element is sandwiched within the foam.

54. The joint spacer according to any one of the previous exemplary embodiments, wherein a reinforcing element is secured within the foam by compression of the foam around the reinforcing element.

55. The joint spacer according to any one of the previous exemplary embodiments, wherein the reinforcing element is sandwiched between two sheets.

56. The joint spacer according to any one of the previous exemplary embodiments, wherein the reinforcing element is sandwiched between the foam and the sheet.

57. The joint spacer according to any one of the previous exemplary embodiments, wherein the reinforcing element comprises a suture.

58. The joint spacer according to any one of the previous exemplary embodiments, wherein the reinforcing element comprises radiopacity.

59. The joint spacer according to any one of the previous exemplary embodiments, wherein the reinforcing element comprises a polymer cable.

60. The joint spacer according to any one of the previous exemplary embodiments, wherein the reinforcing element comprises a textile.

61. The joint spacer according to any one of the previous exemplary embodiments, wherein the reinforcing element comprises a textile and the textile comprises a mesh.

62. The joint spacer according to any one of the previous exemplary embodiments, wherein the reinforcing element comprises a textile and the textile comprises a knit or a woven.

63. The joint spacer according to any one of the previous exemplary embodiments, wherein the reinforcing element comprises a flat braid.

64. The joint spacer according to any one of the previous exemplary embodiments, wherein the polymer cable comprises a rectangular or oval cross-section.

65. The joint spacer according to any one of the previous exemplary embodiments, wherein the polymer cable comprises a flat shape wherein a ratio between the height and the width of the polymer cable is less than 1, less than 0.5, less than 0.2, or less than 0.1.

66. The joint spacer according to any one of the previous exemplary embodiments, further comprising a second reinforcing element, wherein the reinforcing element and the second reinforcing element cross within the interior of the joint spacer.

67. The joint spacer according to any one of the previous exemplary embodiments, further comprising a second reinforcing element, wherein the reinforcing element and the second reinforcing element are positioned perpendicularly to one another.

68. A method of forming a joint spacer comprising the steps of:
a. compressing a foam into a mold, ; and
b. locking the foam in its compressed state,
   wherein the foam comprises a first articular surface formed at the interface of the foam and the depth of a mold cavity, and second articular surface opposite the first articular surface, wherein the porosity of the foam varies as a function of the thickness of the foam.

69. The method of any one of the previous exemplary embodiment, wherein, prior to compressing the foam into the mold the foam comprises a sheet and optionally a reinforcing element on the first articular surface.

70. The method of any one of the previous exemplary embodiment, wherein, prior to compressing the foam into the mold the foam comprises a sheet and optionally a reinforcing element on the second articular surface.

71. The method of any one of the previous exemplary embodiments, wherein the foam comprises a reinforcing element.

72. The method of any one of the previous exemplary embodiments, wherein the foam comprises a reinforcing element prior to step of compressing the foam.

73. The method of any one of the previous exemplary embodiments, wherein the joint spacer comprises a reinforcing element present at the interface of the sheet and the foam.

74. The method of any one of the previous exemplary embodiments, further comprising the step of physically entangling a sheet to the exposed surface of the foam.

75. The method of any one of the previous exemplary embodiments, further comprising the step of solvent bonding a sheet to the exposed surface of the foam.

76. The method of any one of the previous exemplary embodiments, further comprising the step of separating the foam from the mold, thereby obtaining a joint spacer.

77. The method of any one of the previous exemplary embodiments, wherein the step of locking the foam in its compressed state is performed by heating the foam.

78. The method of any one of the previous exemplary embodiments, wherein the step of locking the foam in its compressed state is performed prior to the step of physically entangling the sheet to the exposed surface of the foam.

The method of any one of the previous exemplary embodiments, wherein the foam comprises a first outward facing surface defined by the thickness of the joint spacer, the first articular surface, and the second articular surface, and a second outward facing surface is opposite the first outward facing surface and defined by the thickness of the joint spacer, the first articular surface, and the second articular surface, and wherein the wherein the foam has a first porosity at a first location and a second porosity, less than the first porosity, at the second outward facing surface, the first location being more proximate the first outward facing surface than the second outward facing surface and the second location being more proximate the second outward facing surface than the first outward facing surface.

79. A joint spacer formed from the method of any one of the previous exemplary embodiments.

80. The method of forming a joint spacer of any one of the previous exemplary embodiments, wherein the obtained joint spacer is the joint spacer of any one of the previous exemplary embodiments.

81. The joint spacer according to any one of the preceding exemplary embodiments, wherein the joint spacer is a meniscus replacement device or a meniscus repair device.

82. A method of treating a patient comprising the steps of:
a. inserting the joint spacer of any one of the previous exemplary embodiments into a joint cavity of a patient.
83. The method of the previous exemplary embodiment, further comprising the step of anchoring a suture positioned within the joint spacer or within a hole in the periphery of the joint spacer to a tissue of the patient.

84. The method of one of the two previous exemplary embodiments, further comprising the steps of positioning a suture in the joint spacer and attaching the suture to a tissue of the patient.

85. The method of the previous exemplary embodiment, wherein the suture in the joint spacer passes through the foam and a sheet, and the suture is retained with the aid of a reinforcing element.

86. The method of the previous exemplary embodiment, wherein the reinforcing element is a textile.

1. A joint spacer comprising a foam, the joint spacer having a first articular surface and a second articular surface, the first articular surface and the second articular defining a thickness of the joint spacer,
wherein a first outward facing surface is defined by the thickness of the joint spacer, the first articular surface, and the second articular surface,
wherein a second outward facing surface is opposite the first outward facing surface and defined by the thickness of the joint spacer, the first articular surface, and the second articular surface,
wherein the foam has a first porosity at a first location and a second porosity, less than the first porosity, at a second location, the first location being more proximate the first outward facing surface than the second outward facing surface and the second location being more proximate the second outward facing surface than the first outward facing surface.

2. The joint spacer according to claim 1, wherein the first location is at the first outward facing surface and the second location is at the second outward facing surface.

3. The joint spacer according to claim 1, wherein the thickness of the joint spacer is greater at the first location than at the second location.

4. The joint spacer according to claim 1, wherein the porosity of the joint spacer varies in relation to its thickness.

5. The joint spacer according to claim 1, wherein the joint spacer has uniform porosity between the first articular surface and the second articular surface at all locations of equal thickness.

6. The joint spacer of claim 1, wherein the second articular external surface is substantially planar.

7. The joint spacer according to claim 1, wherein the joint spacer comprises a planar portion wherein the distance between the first articular surface and the second articular surface is about constant.

8. The joint spacer of claim 1, wherein the porosity is about constant throughout the planar portion.

9. The joint spacer according to claim 1, wherein the foam is a single piece of foam.

10. The joint spacer according to claim 1, wherein the foam is under compression least one location.

11. The joint spacer according to claim 1, wherein the foam is under compression at all locations.

12. The joint spacer according to claim 1, wherein the joint spacer the foam is a single piece of foam under compression, and wherein the amount of compression varies with the thickness of the joint spacer.

13. The joint spacer according to claim 1, wherein the foam is an open cell polyurethane foam.

14. The joint spacer according to claim 1, wherein the joint spacer comprises a sheet adhered to the foam.

15. The joint spacer according to claim 1, wherein the second articular surface comprises a polyurethane sheet.

16. The joint spacer according to claim 1, wherein the foam and the sheet are physically entangled with one another.

17. The joint spacer of claim 1, wherein the sheet is a polyurethane and the foam is a polyurethane.

18. The joint spacer of claim 1, wherein the foam and the sheet are each biostable.

19. The joint spacer of claim 1, wherein the foam comprises a polycarbonate polyurethane and the sheet comprises a polycarbonate polyurethane.

20. The joint spacer of claim 1, wherein the sheet has a thickness of from 10 microns to 1000 microns.

21. The joint spacer according to claim 1, further comprising a reinforcing element embedded in the joint spacer.

22. The joint spacer according to claim 21, wherein the reinforcing element is secured at an interface of the foam and the sheet by a bonding of the foam and the sheet around the reinforcing element.

23. The joint spacer according to claim 1, wherein a reinforcing element is secured within the foam by compression of the foam around the reinforcing element.

24. The joint spacer according to claim 21, wherein the reinforcing element comprises a suture, a polymer cable, or a textile.

25. A method of forming a joint spacer comprising the steps of:
a. compressing a foam into a mold; and
b. locking the foam in its compressed state,
wherein the foam comprises a first articular surface formed at the interface of the foam and the depth of a mold cavity, and second articular surface opposite the first articular surface, wherein the porosity of the foam varies as a function of the thickness of the foam.

26. The method of claim 1, wherein the step of locking the foam in its compressed state is performed by heating the foam.

27. The method of claim 1, further comprising the step of physically entangling a sheet to an exposed surface of the foam.

28. The method of claim 1, further comprising the step of physically entangling a sheet to an exposed surface of the foam and thereby securing a reinforcing element between the sheet at the foam.

29. The joint spacer or method according to claim 1, wherein the reinforcing element is a textile.
